(19) United States
(12) Patent Application Publication
(10) Pub. No.: US 2022/0118189 A1
(43) Pub. Date: Apr. 21, 2022

(54) ADJUSTABLE DOSE DRUG DELIVERY SYSTEM

(71) Applicant: Beloteca Investment Fund 1, LLC, San Diego, CA (US)

(72) Inventor: Lawrence J. Kelly, Fairfield, OH (US)

(21) Appl. No.: 17/422,194

(22) PCT Filed: Jan. 14, 2020
(86) PCT No.: PCT/US20/13420

§ 371 Date: Jul. 11, 2021

Related U.S. Application Data
(60) Provisional application No. 62/792,972, filed on Jan. 16, 2019.

(51) Int. Cl. A61M 5/315; A61M 5/32

(52) U.S. Cl. CPC A61M 5/3156; A61M 5/3202; A61M 5/31505; A61M 2005/3125; A61M 5/31591; A61M 5/3155; A61M 5/31538

(57) ABSTRACT

A drug delivery system is provided that has an adjustable dose mechanism allowing setting and locking the dose then administration of the set dose. A method for administering a medicament using the drug delivery system is also provided.

### CROSS-REFERENCE TO RELATED APPLICATIONS

This application claims the benefit of U.S. Provisional Application No. 62/792,972. filed Jan. 16, 2019, and incorporated by reference herein in its entirety.

### BACKGROUND OF THE INVENTION

### (1) Field of the Invention

The present application generally relates to drug administration systems. More specifically, systems having an adjustable dose mechanism allowing setting and locking the dose then administration of the set dose.

### (2) Description of the Related Art

A system for home administration of a drug has several preferred characteristics. The system should have simple, foolproof procedures for administering the drug. The system also preferably has a dosage adjustment mechanism that allows for the dosage to be set depending on the needs of the patient. The system can also have a mechanism allowing the set dosage to be locked, e.g., by a pharmacist. See U.S. Pat. Nos. 5,462,740; 5,578,011; 5,632,733; 6,090,077; 6,572,584; 7,329,241; and 7,351,224, and PCT Patent Publication WO 2018/085759. Provided is a drug delivery system having the above characteristics.

### BRIEF SUMMARY OF THE INVENTION

Provided is an adjustable dose drug delivery system. The system comprises a plunger, a syringe body, an inner housing, an outer housing, a stop sleeve, and a lock collar, wherein

the plunger comprises a stopper at a plunger distal end, a plunger proximal end, and a plunger stem extending from the plunger distal end to the plunger proximal end;

the syringe body comprises a cylinder, and further comprises a syringe tip at a syringe body distal end and an open syringe body proximal end,
wherein the plunger is slidably deposed by inserting the stopper into the open proximal end of the syringe body and pushing the plunger flange toward the syringe body distal end;

the inner housing comprises a cylinder, and further comprises an inner housing proximal end and an inner housing distal end and substantially encloses the syringe body and is affixed thereto, wherein the inner housing further comprises
an upper flange below the engagement pits,
a lower flange between the upper flange and the inner housing distal end, with an inter-flange area between the upper flange and lower flange, and
an administration tip distal to the lower flange that is hollow and accommodates, at its proximal end, the syringe tip;

the outer housing comprises a cylinder that encompasses the inner housing from above the lower flange to above the mechanism that engages the stop sleeve, the outer housing further comprising an outer housing proximal end and an outer housing distal end, wherein the outer housing further comprises
an outer housing distal region adjacent to the inter-flange area of the inner housing, wherein the outer housing distal region
   has a narrower circumference than the outer housing proximal end, and
   comprises a lock collar actuating mechanism that, when engaged, prevents rotation of the outer housing relative to the inner housing;
a dosage window displaying dosage information present on an outer surface of the stop sleeve; and
a stop sleeve engagement mechanism that engages the stop sleeve to prevent the stop sleeve from further movement when a designated dosage is dispensed;

the stop sleeve comprises a cylinder with a stop sleeve proximal end and a stop sleeve distal end, wherein the stop sleeve is deposed between the outer housing and the inner housing above the upper flange, the stop sleeve further comprising
a mechanism that engages the stop sleeve engagement mechanism on the outer housing, and
markings near the stop sleeve distal end indicating dosage settings; and

the lock collar encircles a portion of the outer housing distal region and engages with the lock collar actuating mechanism when the lock collar is slid toward the upper flange.

Also provided is a method of administering a medicament using the above adjustable dose drug delivery system. The method comprises hold the outer housing proximal to the outer housing distal region, grasp the lower flange and turn the lower flange relative to the outer housing until a desired dosage is indicated in the dosage window;

slide the lock collar toward the upper flange to engage the lock collar actuating mechanism; insert the administration tip into a bodily orifice of a patient in need of the medicament;

dispense the medicament through the administration tip by pushing the plunger toward the syringe body distal end until the plunger cannot be pushed any further.

### BRIEF DESCRIPTION OF THE DRAWINGS

In accordance with the present invention,

FIG. 1 is a perspective view of an adjustable dose drug delivery system.

FIG. 2 is an exploded view of an adjustable dose drug delivery system.

FIG. 3 is a transparent view of a plunger inserted into a syringe body.

FIG. 4 is a perspective view of a syringe body with plunger inserted therein with an inner housing.

FIG. 5 is a transparent view of a syringe body and plunger inside an inner housing.

FIG. 6 is a transparent view of a syringe body and plunger inside an inner housing with a stop sleeve and snap top.

FIG. 7 is a transparent view of a syringe body, plunger, inner housing, stop sleeve, snap top and outer housing.

FIG. 8 is a transparent view of a syringe body, plunger, inner housing, stop sleeve, snap top and outer housing, with an exploded view of a lock collar.

FIG. 9 is a transparent view of a syringe body, plunger, inner housing, stop sleeve, snap top, outer housing and lock collar.

FIG. 10 is a transparent view of a syringe body, plunger, inner housing, stop sleeve, snap top, outer housing, lock collar and seal pin.

FIG. 11 is a transparent view of a syringe body, plunger, inner housing, stop sleeve, snap top, outer housing, lock collar and protective cap.

FIG. 12 is a close-up transparent view of the distal portion of an outer housing and an inner housing, with a lock collar, seal pin and protective cap.

FIG. 13 is a close-up view of the distal portion of an outer housing, with a lock collar and protective cap.

FIG. 14 is a perspective view of the inside of a half of an outer housing with a lock collar half.

FIG. 15 is a perspective view of the inside of a half of an outer housing with a lock collar half.

FIG. 16 is a perspective view of a protective cap, snap top, stop sleeve and inner housing with a cutaway of an outer housing.

FIG. 17 is a closeup of a stop sleeve, snap top and a portion of an inner housing.

FIG. 18 is a closeup of a protective cap with a portion of the inside of an outer housing with a lock collar half.

FIG. 19 is a cutaway of an outer housing over an inner housing.

### DETAILED DESCRIPTION OF THE INVENTION

As used herein, the singular forms “a”, “an” and “the” are intended to include the plural forms as well, unless the context clearly indicates otherwise. Additionally, the use of “or” is intended to include “and/or”, unless the context clearly indicates otherwise.

As used herein, “distal” is toward the end of the administrative tip; “proximal” is toward the portion of the plunger that is furthest from the stopper.

Provided is an adjustable dose drug delivery system. The system comprises a plunger, a syringe body, an inner housing, an outer housing, a stop sleeve, and a lock collar, wherein

the plunger comprises a stopper at a plunger distal end, a plunger proximal end, and a plunger stem extending from the plunger distal end to the plunger proximal end;

the syringe body comprises a cylinder, and further comprises a syringe tip at a syringe body distal end and an open syringe body proximal end comprising a syringe body flange,
wherein the plunger is slidably deposed by inserting the stopper into the open proximal end of the syringe body and pushing the plunger flange toward the syringe body distal end;

the inner housing comprises a cylinder, and further comprises an inner housing proximal end and an inner housing distal end and substantially encloses the syringe body, wherein the inner housing further comprises
an inner housing portion of a mechanism that engages the inner housing and stop sleeve to limit movement of the stop sleeve after the plunger is pushed toward the syringe body distal end in accordance with a dosage setting;
an upper flange below the mechanism that engages the inner housing and stop sleeve,
a lower flange between the upper flange and the inner housing distal end, with an inter-flange area between the upper flange and lower flange, and
an administration tip distal to the lower flange that is hollow and accommodates, at its proximal end, the syringe tip;

the outer housing comprises a cylinder that encompasses the inner housing from above the lower flange to above the mechanism that engages the stop sleeve, the outer housing further comprising an outer housing proximal end and an outer housing distal end, wherein the outer housing further comprises
an outer housing distal region adjacent to the inter-flange area of the inner housing, wherein the outer housing distal region
   has a narrower circumference than the outer housing proximal end, and
   comprises a lock collar actuating mechanism that, when engaged, prevents rotation of the outer housing relative to the inner housing;
a dosage window displaying dosage information present on an outer surface of the stop sleeve; and
a stop sleeve engagement mechanism that engages the stop sleeve to prevent the stop sleeve from further movement when a designated dosage is dispensed;

the stop sleeve comprises a cylinder with a stop sleeve proximal end and a stop sleeve distal end, wherein the stop sleeve is deposed between the outer housing and the inner housing above the upper flange, the stop sleeve further comprising
a stop sleeve portion of a mechanism that engages the inner housing and stop sleeve;
a mechanism that engages the stop sleeve engagement mechanism on the outer housing, and
markings near the stop sleeve distal end indicating dosage settings; and

the lock collar encircles a portion of the outer housing distal region and engages with the lock collar actuating mechanism when the lock collar is slid toward the upper flange.

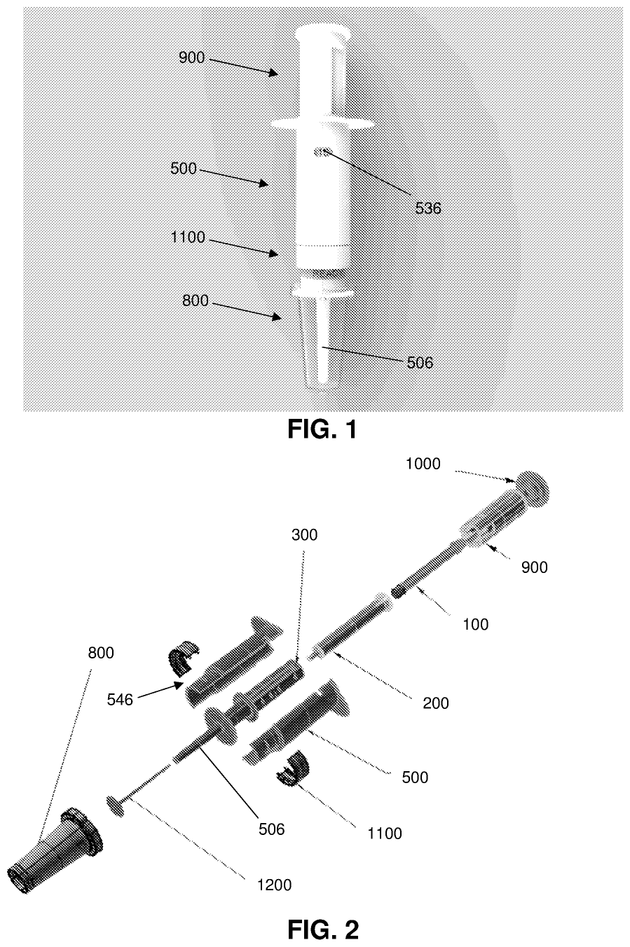

Various nonlimiting embodiments of the system are depicted in the figures. One such embodiment is depicted in FIG. 1, showing a stop sleeve 900, an outer housing 500, a lock collar 1100, a protective cap 800 that protects an administration tip 506 when the system is not in use, and a dosage window 536. An exploded illustration of the system, FIG. 2, reveals additional features and relationships between features. A snap top 1000 may be joined to a plunger 100 to provide comfortable support for a finger or thumb to administer a medicament by pushing the plunger toward the distal end of the inside of a syringe body 200. In some embodiments, the snap top 1000 is originally separated from the plunger in order to slide the stop sleeve 900 over the plunger 100 during manufacture of the system. In other embodiments, the snap top is integrated with the plunger at manufacture. The outer housing 500 envelops the inner housing 300, which has the administration tip 506 at its distal end, which may be covered with the protective cap 800. In various embodiments, a channel extending through the length of the administration tip 506, through which a medicament is administered, has a seal pin 1200 inserted therein when the system is not in use. The distal region of the outer housing 546 is enveloped by the lock collar 1100.

Where it may be convenient, any of the parts of the system may be manufactured in parts (e.g., in halves), as shown with the outer housing 500 and the lock collar 1100, which can be assembled during production of the system.

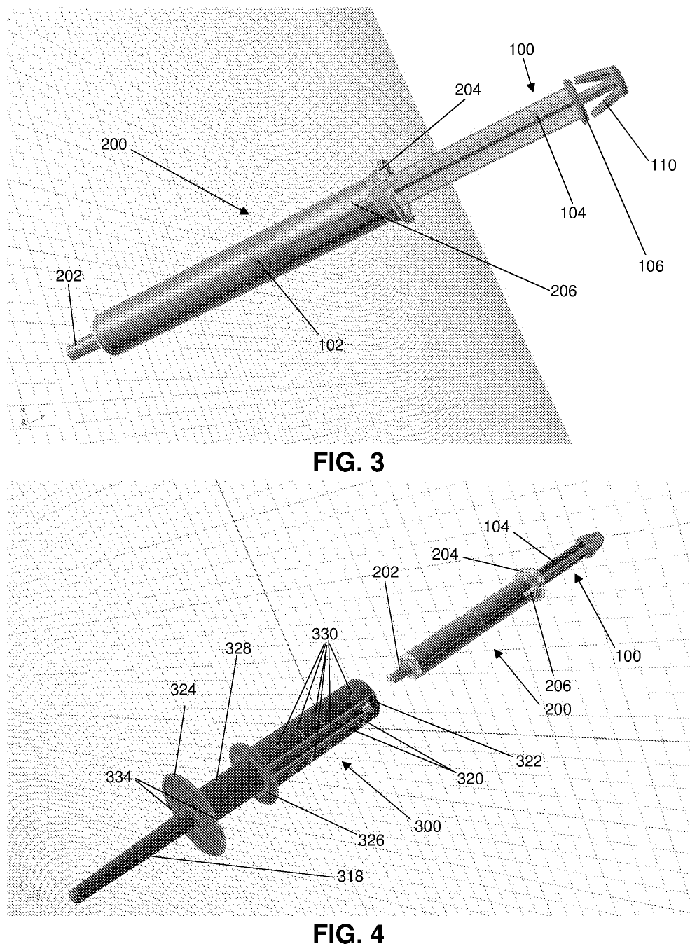

FIG. 3 provides details of a plunger 100 and syringe body 200 featured in some embodiments of the invention. The illustrated plunger 100 comprises a stopper 102 at its distal end, a plunger stem 104, a plunger flange 204, and a cap connector 100. The cap connector 100 is designed to couple with a snap cap 1000 and rest on the plunger flange 204 when the snap cap 1000 is snapped onto the cap connector 100. The interior of the syringe body 200 can hold a medicament before administration. The distal end of the syringe body 200 has a syringe tip 202, through which a substance (e.g., a medicament) in the syringe body 200 is dispensed into the administration tip 318 when the plunger 100 is pushed through the syringe body 200. The proximal end of the syringe body 200 has a syringe body flange 204, and a syringe body tab 206. These features engage the inner housing as described below.

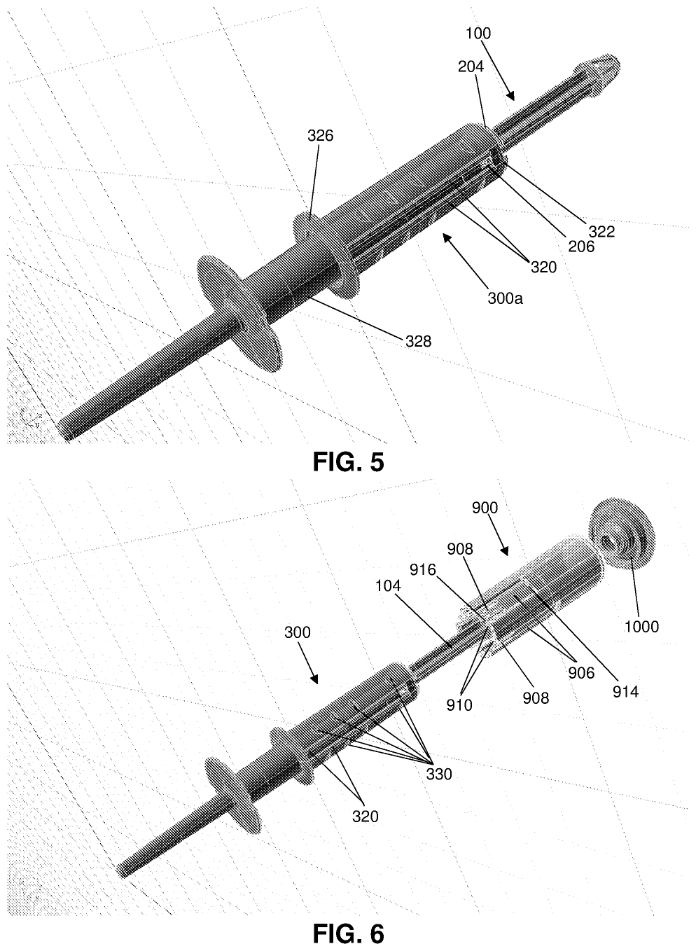

The interaction of the syringe body 200 with the inner housing 300 is shown in FIG. 4 and FIG. 5. The illustrated embodiment shows an inner housing 300 having a lower flange 324 with lower flange indentations 334 that engages the protective cap 800 to retain the protective cap 800 on the lower flange 324 to protect the administration tip 318. The inner housing 300 also has an upper flange 326, an inter-flange area 328, stop sleeve engagement rails 320 and a plurality of stop sleeve engagement pits 330 that are circumferentially and vertically deposed on the inner housing between the inner housing proximal end and the inner housing distal end, above the upper flange 326. The functions of these external portions of the inner housing 300 are discussed below. The syringe body 200, with a plunger 100 inserted therein, is deposed inside the inner housing 326 such that the inner housing 326 substantially encloses the syringe body 200.

In the illustrated embodiments, the syringe body 200 is inserted into the inner housing 326 until an attachment tab 322 at the proximal end of the inner housing 300 engages a syringe body tab 206 at the proximal end of the syringe body 200, joining the syringe body 200 with the inner housing 300 such that the proximal end of the inner housing 300 abuts the bottom of the syringe body flange 204. There may be more than one (e.g., 2, 3 or 4) syringe body tab 206 and attachment tab 322.

FIG. 6 shows the relationship of the inner housing 300 with the stop sleeve 900 in the illustrated embodiment. The stop sleeve 900 includes stop sleeve slots 906 comprising indentations in the stop sleeve, the stop sleeve slots 906 deposed at the stop sleeve distal end and extending toward the stop sleeve proximal end for varying distances depending on a specific dosage corresponding to each stop sleeve slot (further discussed below) 906, and stop sleeve tongues 908, at the end of each stop sleeve slot 906. The stop sleeve tongue 908 has an inner knob 916 protruding inside of a distal end of the stop sleeve tongue 908 that engages the engagement pits 330 when the plunger 100 and stop sleeve 900 are pushed distally, limiting movement of the stop sleeve 900 toward the inner housing 300 proximal end. In some embodiments, the stop sleeve tongue 908 and engagement pits 330 are not present.

The stop sleeve 900 also has inner housing engagement guides 910 that engage the elevated stop sleeve engagement rails 320 to join the inner housing 300 with the stop sleeve 900 to rotate together. There may be more than one (e.g., 2, 3, or 4) set of inner housing engagement guides 910 and stop sleeve engagement rails 320 circumferentially deposed on the stop sleeve 900 and inner housing 300.

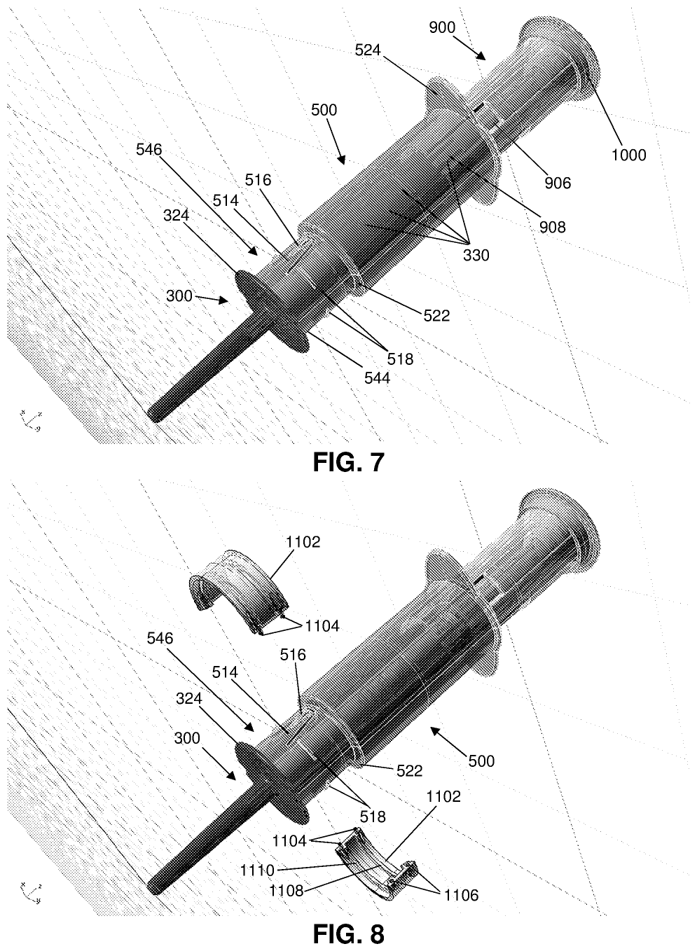

The outer housing 500 and its relationship to the stop sleeve 900 and inner housing 300 is shown in FIG. 7. In the illustrated embodiment, the distal end of the outer housing 544 rests on the lower flange of the inner housing 324. The outer housing distal region 546 is indented, forming an outer housing ridge 522 on its proximal edge, and comprises at least one outer housing flap 514 comprising a lock collar engagement tab 516, a lock collar guide rail 518, which are components that engage the lock collar 1100. The illustrated outer housing has an outer housing flange 524 at its proximal end, which provides a finger hold when a medicament is administered by pushing on the snap top 1000 in a distal direction, which also moves the stop sleeve 900, and the plunger 100 distally. In some embodiments, a stop sleeve tongue 908 and engagement pits 330 are present. In those embodiments, the stop sleeve tongue 908 sequentially engages the engagement pits 330 through its inner knob 916, preventing proximal movement of the stop sleeve 900, and the plunger 100 and snap top 1000 joined thereto.

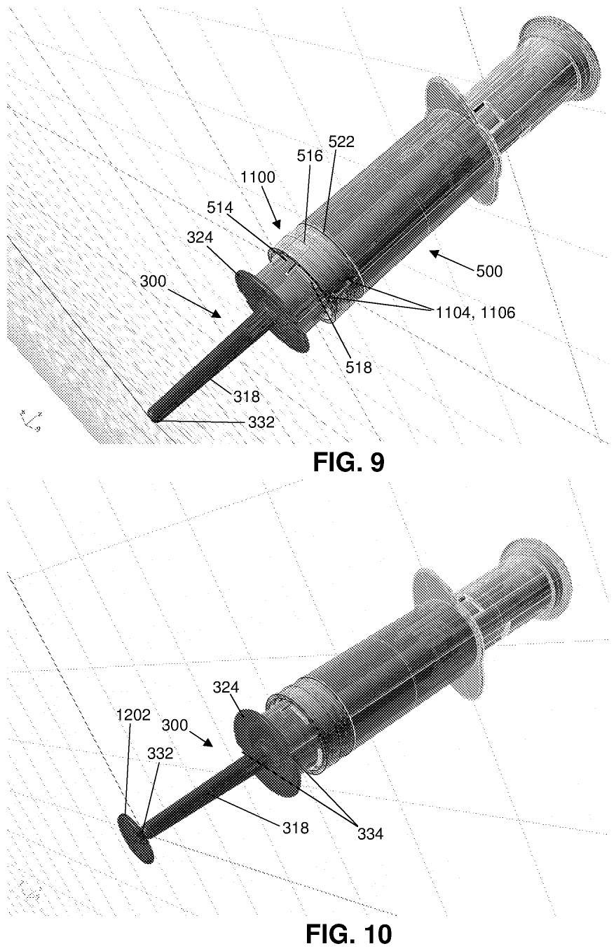

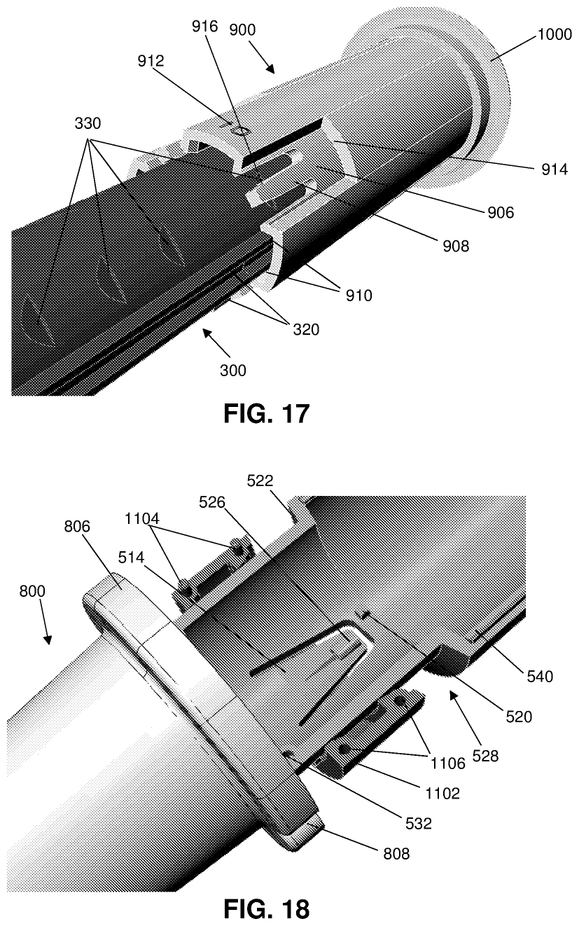

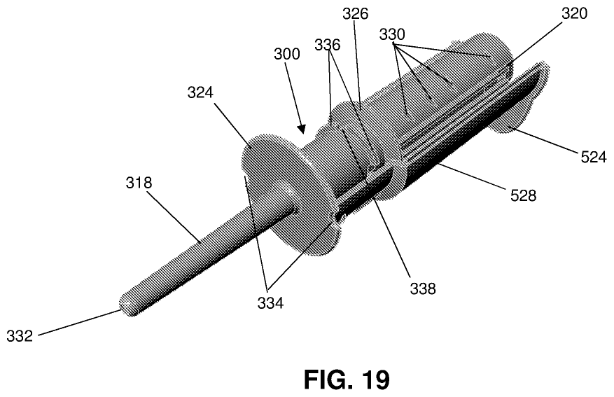

FIG. 8 and FIG. 9 show the lock collar 1100 of the illustrated embodiment, and its engagement with the outer housing distal region 546. The lock collar 1100 can be manufactured in halves 1102, which are joined during assembly of the system. In some embodiments, the lock collar halves 1102 have lock collar assembly pegs 1104 which are inserted into lock collar assembly holes 1106 during assembly. Before use, the lock collar 1100 encircles the distal portion of the outer housing distal region 546. After the dosage is set, by turning the outer housing 500 relative to the stop sleeve 900 until the designated dosage, as indicated by the dosage marking 912 on a distal portion of the stop sleeve 900, as shown in FIG. 17, appears in the dosage window 536 of the outer housing 500, as shown in FIG. 14, the dosage setting is locked by sliding the lock collar 1100 to the outer housing ridge 522 until a lock collar upper shelf 1108, on the inside of the lock collar 1100 engages the lock collar engagement tab 516 on the outer housing flap 514. The lock collar lower shelf 1110, distal to the lock collar upper shelf 1108, then rests on the lock collar guide rail 518 on the outer housing 500. When the lock collar 1100 is so engaged, a lock collar engagement tab 516 on the inside of the outer housing 500, as shown in FIG. 14, is pushed inwardly to cause the outer housing flap inner tab 526, as shown in FIGS. 12, 14, and 15, to interfere with the rotation of the outer housing 500 relative to the inner housing 300 by entering a gap in the dosage lock railing 336 of the inner housing 300, as shown in FIG. 16 and FIG. 19.

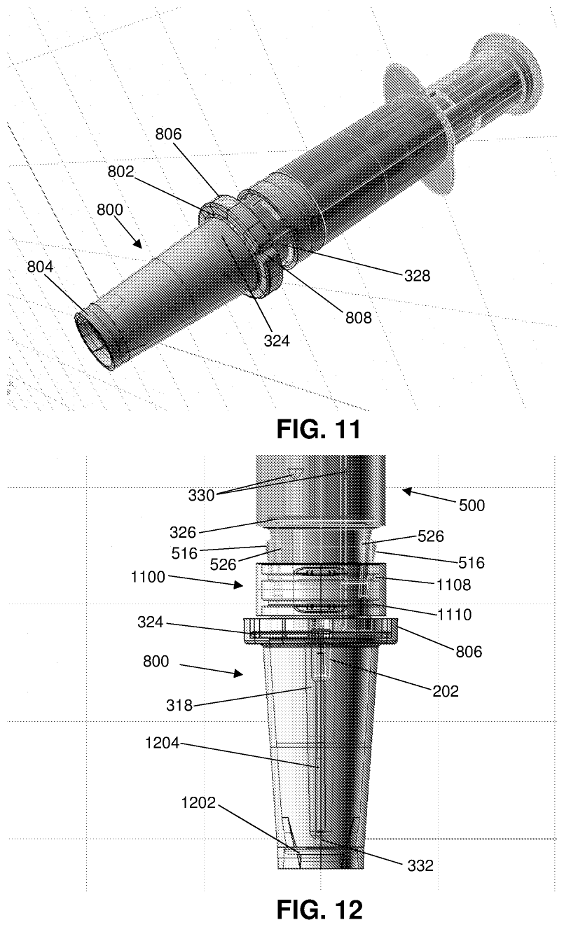

FIG. 10 and FIG. 11 illustrate aspects of the distal portion of an embodiment of the system, showing the seal pin top 1202 at the dispensing hole 332 at the distal point of the administration tip 318. The lower flange 324 has indentations 334 that match indentations 808 in the cap connector 806 at the proximal end of the protective cap 802, such that the protective cap 800 stays on the lower flange 324 until manually removed.

FIG. 12 shows the distal portion of the system in transparent view before engaging the lock collar 1100, further showing the relationship between various parts, including the upper flange 326, the lower flange 324, the lock collar engagement tab 516, the outer housing flap inner tab 526, the lock collar upper shelf 1108, the lock collar lower shelf 1110, the protective cap 800, the cap connector 806, the syringe tip 202, the administration tip 318, the dispensing hole 332, the seal pin top 1202, and the seal pin stem 1204.

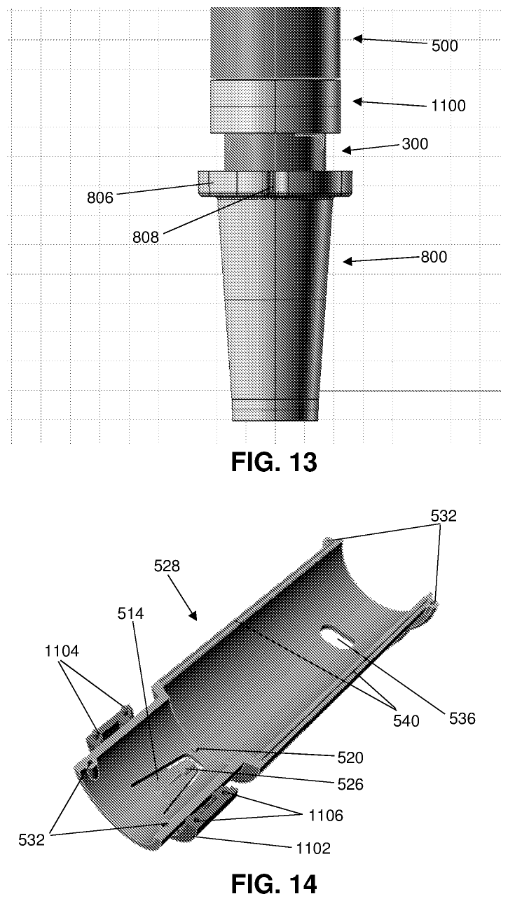

The distal portion of the system after the lock collar 1100 is engaged is shown in FIG. 13, showing, inter alia, the protective cap 800, with its cap connector 806 and cap connector indentations 808.

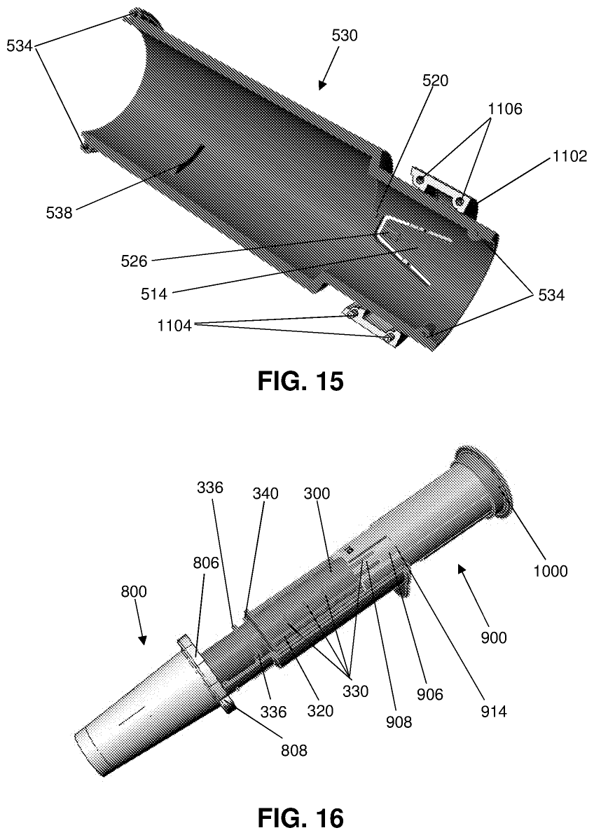

FIG. 14 and FIG. 15 show two halves of the outer housing 500, outer housing half A 528, and outer housing half B 530. The outer housing 500 may be constructed in parts, e.g., halves, as illustrated, for example with assembly holds 532 and assembly pegs 534 and outer housing assembly alignment ridges 540, the latter feature used to ultrasonically “weld” the two outer housing halves 528, 530 together. The outer housing rotation tabs 520 interacts with the inner housing rotation tab 340, shown in FIG. 16, to allow the inner housing 300 to turn 180 degrees to set the doses.

The dosage setting mechanism is shown in FIG. 14, FIG. 15 and FIG. 16, the latter figure showing the system with part of the outer housing 500 omitted. As previously discussed, the dosage is set by turning the outer housing 500 relative to the stop sleeve 900 until the designated dosage, as indicated by the dosage marking 912 on a distal portion of the stop sleeve 900, as shown in FIG. 17, appears in the dosage window 536 of the outer housing half A 528. This aligns the stop sleeve slot 906 corresponding to the set dosage with the stop sleeve slot engagement ridge 538 on outer housing half B 530. When the plunger 100 is pushed distally along with the stop sleeve 900, the stop sleeve 900 travels along the inside of the outer housing 500, the stop sleeve slot 906 corresponding to the set dosage traveling along outer housing half B 530 until the stop sleeve slot proximal end 914 abuts against the stop sleeve slot engagement ridge 538, preventing further distal movement, and causing the administration of the precise dosage that was set. A closeup view of relevant features at the proximal portion of the system is shown in FIG. 17, including the dosage marking 912 on the stop sleeve, and the stop sleeve slot proximal end 914. The stop sleeve tongue 908, the stop sleeve tongue inner knob 916 and the stop sleeve tongue engagement pits 330 are also shown, which are present in some embodiments, and prevent movement of the plunger 100 and stop sleeve 900 in the proximal direction, as previously discussed.

Additional views of various features of the illustrated embodiments described above are shown in FIG. 18 and FIG. 19.

Although the illustrated embodiments provide for a long administration tip 318, which is particularly suitable for rectal administration, the system of the present invention is also suitable to be used with needles, where a needle is utilized instead of the administration tip 318. The needle could be used for any administration that uses a needle, e.g., intravenous, subdermal, intramuscular, intraperitoneal, etc., as those administration means are known in the art.

In certain specific embodiments, the drug delivery system has an administration tip suitable for rectal administration, e.g., administration tip 318, and the drug is a benzodiazepine, for example diazepam. Such a system is suitable for treatment of, e.g., epilepsy.

Also provided is a method of administering a medicament using the above adjustable dose drug delivery system. The method comprises

hold the outer housing proximal to the outer housing distal region, grasp the lower flange and turn the lower flange relative to the outer housing until a desired dosage is indicated in the dosage window;

slide the lock collar toward the upper flange to engage the lock collar actuating mechanism; insert the administration tip into a bodily orifice of a patient in need of the medicament;

dispense the medicament through the administration tip by pushing the plunger toward the syringe body distal end until the plunger cannot be pushed any further.

In some of these embodiments, the method further comprises after dispensing the medicament,

pull the plunger in a proximal direction until the plunger is removed from the syringe body;

replace the plunger into the syringe body and push the plunger toward the syringe body distal end until all of the medicament is dispensed through the administration tip.

In some of these methods, the medicament is benzodiazepine and the bodily orifice is a rectal opening.

In certain of these embodiments, the turning of the lower flange relative to the outer housing and the sliding of the lock collar toward the upper flange is performed by a pharmacist who is not the patient.

In additional embodiments of these methods, the adjustable dose drug delivery system further comprises the seal pin inserted into the administration tip and the protective cap,

the lower flange is grasped and turned by grasping and turning the administration tip, and

the protective cap and the seal pin is removed before the administration tip is inserted into a bodily orifice of the patient.

In specific embodiments as illustrated in the figures, an adjustable dose drug delivery system is provided. The system comprises a plunger, a syringe body, an inner housing, an outer housing, a stop sleeve, a lock collar, a seal pin, and a protective cap, wherein

the plunger comprises a stopper at a plunger distal end, a plunger proximal end, and a plunger stem extending from the plunger distal end to the plunger proximal end, the plunger proximal end further comprises a cap connector and a snap top, wherein
the snap top is a disk with a central fastener that joins with the cap connector to affix the snap top to the plunger proximal end;

the syringe body comprises a cylinder, and further comprises a syringe tip at a syringe body distal end and an open syringe body proximal end comprising a syringe body flange,
wherein the plunger is slidably deposed by inserting the stopper into the open proximal end of the syringe body and pushing the plunger flange toward the syringe body distal end;

the inner housing comprises a cylinder, and further comprises an inner housing proximal end and an inner housing distal end and encloses the syringe body and is affixed thereto, wherein the inner housing further comprises
an upper flange below the engagement pits,
a lower flange between the upper flange and the inner housing distal end, with an inter-flange area between the upper flange and lower flange, and an administration tip distal to the lower flange that is hollow and accommodates, at its proximal end, the syringe tip;

the outer housing comprises a cylinder that encompasses the inner housing from above the lower flange to above the mechanism that engages the stop sleeve, the outer housing further comprising an outer housing proximal end, an outer housing distal end, wherein the outer housing further comprises
an outer housing distal region adjacent to the inter-flange area of the inner housing, wherein the outer housing distal region
   has a narrower circumference than the outer housing proximal end, and
   comprises a lock collar actuating mechanism that, when engaged, prevents rotation of the outer housing relative to the inner housing;
a dosage window displaying dosage information present on an outer surface of the stop sleeve; and
   the stop sleeve comprises a cylinder with a stop sleeve proximal end and a stop sleeve distal end, wherein the stop sleeve is deposed between the outer housing and the inner housing above the upper flange,

the stop sleeve further comprising
markings near the stop sleeve distal end indicating dosage settings;
a plurality of stop sleeve slots comprising indentations in the stop sleeve, the stop sleeve slots deposed at the stop sleeve distal end and extending toward the stop sleeve proximal end for varying distances depending on a specific dosage corresponding to each stop sleeve slot; and
a stop sleeve slot engagement ridge protruding from an inside surface of the outer housing, wherein the stop sleeve slot engagement ridge engages with a stop sleeve slot proximal end to limit movement of the stop sleeve and attached plunger in accordance with the dosage setting;

the lock collar encircles a portion of the outer housing distal region and engages with the lock collar actuating mechanism when the lock collar is slid toward the upper flange,

wherein the lock collar actuating mechanism comprises
an outer housing flap in the outer housing distal region, the outer housing flap further comprising (a) a lock collar engagement tab the protrudes outward on the outer housing flap, and (b) an outer housing flap inner tab protruding from an inside surface of the outer housing flap;
a lock collar upper shelf comprising a ledge protruding from an inside of the lock collar on a proximal area of the lock collar; and
a dosage lock railing comprising a ledge protruding from a proximal area of the inter-flange area of the inner housing, wherein the dosage lock railing has a gap that accommodates the outer housing flap inner tab, wherein
when the lock collar is slid toward the upper flange, the ledge of the lock collar upper shelf pushes the lock collar engagement tab, directing the outer housing flap inwardly, which causes the outer housing flap inner tab to enter the gap in the dosage lock railing, preventing rotation of the outer housing relative to the inner housing;

the seal pin comprises
a seal pin stem comprising a long, thin needle that can be inserted into the administration tip hollow portion through a dispensing hole at a distal end of the administration tip; and
a seal pin top at a distal end of the seal pin stem, wherein the seal pin top prevents complete insertion of the seal pin into the administration tip; and

the protective cap encloses the administration tip and is affixed to the lower flange,

the adjustable dose drug delivery system further comprising:
components joining the inner housing and the stop sleeve to rotate together, comprising
a stop sleeve engagement rail comprising an elevated rail on the inner housing that substantially extends from the inner housing upper flange to the inner housing proximal end; and
an inner housing engagement guide comprising a protruding area at the stop sleeve distal end that accommodates the stop sleeve engagement rail;

a lock collar lower shelf comprising a ledge protruding from an inside of the lock collar on a distal area of the lock collar; and

a lock collar guide rail comprising a ledge in the outer housing distal region, wherein

the lock collar lower shelf rests on the lock collar guide rail prior to engagement of the lock collar actuating mechanism, wherein

the dosage is set by turning the outer housing relative to the stop sleeve, inner housing, syringe body and plunger until a desired dosage marked on the stop sleeve is indicated in the dosage window;

the dosage setting is locked by sliding the lock collar toward the upper flange until the lock collar actuating mechanism is engaged; and

the set dosage of a substance present in the syringe body is expelled through a dispensing hole at a distal end of the administration tip.

Also provided is a method of administering a medicament using the adjustable dose drug delivery system as described immediately above and as illustrated in the figures. The method comprises

hold the outer housing proximal to the outer housing distal region, grasp the protective cap and turn the protective cap relative to the outer housing until a desired dosage is indicated in the dosage window;

slide the lock collar toward the upper flange to engage the lock collar actuating mechanism;

remove (a) the protective cap from the lower flange, and (b) the seal pin from the administration tip;

insert the administration tip into a bodily orifice of a patient in need of the medicament; and

dispense the medicament through the administration tip by pushing the plunger toward the syringe body distal end until the plunger cannot be pushed any further.

In various embodiments, the method further comprises, after dispensing the medicament,

pull the plunger in a proximal direction until the plunger is removed from the syringe body; and

replace the plunger into the syringe body and push plunger toward the syringe body distal end until all of the medicament is dispensed through the administration tip.

In certain embodiments of this method, the medicament is benzodiazepine and the bodily orifice is a rectal opening.

In other embodiments, the turning of the protective cap relative to the outer housing and the sliding of the lock collar toward the upper flange is performed by a pharmacist who is not the patient.

In view of the above, it will be seen that several objectives of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification are hereby incorporated by reference. The discussion of the references herein is intended merely to summarize the assertions made by the authors and no admission is made that any reference constitutes prior art. Applicants reserve the right to challenge the accuracy and pertinence of the cited references.

What is claimed is:

1. An adjustable dose drug delivery system comprising:
a plunger, a syringe body, an inner housing, an outer housing, a stop sleeve, and a lock collar, wherein
the plunger comprises a stopper at a plunger distal end, a plunger proximal end, and a plunger stem extending from the plunger distal end to the plunger proximal end;
the syringe body comprises a cylinder, and further comprises a syringe tip at a syringe body distal end and an open syringe body proximal end,
wherein the plunger is slidably deposed by inserting the stopper into the open proximal end of the syringe body and pushing the plunger flange toward the syringe body distal end;
the inner housing comprises a cylinder, and further comprises an inner housing proximal end and an inner housing distal end and substantially encloses the syringe body, wherein the inner housing further comprises
an inner housing portion of a mechanism that engages the inner housing and stop sleeve to limit movement of the stop sleeve after the plunger is pushed toward the syringe body distal end in accordance with a dosage setting;
an upper flange below the mechanism that engages the inner housing and stop sleeve,
a lower flange between the upper flange and the inner housing distal end, with an inter-flange area between the upper flange and lower flange, and
an administration tip distal to the lower flange that is hollow and accommodates, at its proximal end, the syringe tip;
the outer housing comprises a cylinder that encompasses the inner housing from above the lower flange to above the mechanism that engages the stop sleeve, the outer housing further comprising an outer housing proximal end and an outer housing distal end, wherein the outer housing further comprises
an outer housing distal region adjacent to the inter-flange area of the inner housing, wherein the outer housing distal region
has a narrower circumference than the outer housing proximal end, and
comprises a lock collar actuating mechanism that, when engaged, prevents rotation of the outer housing relative to the inner housing; and
a dosage window displaying dosage information present on an outer surface of the stop sleeve;
the stop sleeve comprises a cylinder with a stop sleeve proximal end and a stop sleeve distal end, wherein the stop sleeve is deposed between the outer housing and the inner housing above the upper flange, the stop sleeve further comprising
a stop sleeve portion of a mechanism that engages the inner housing and stop sleeve; and
markings near the stop sleeve distal end indicating dosage settings; and
the lock collar encircles a portion of the outer housing distal region and engages with the lock collar actuating mechanism when the lock collar is slid toward the upper flange.

2. The adjustable dose drug delivery system of claim 1, wherein
the dosage is set by turning the outer housing relative to the stop sleeve, inner housing, syringe body and plunger until a desired dosage marked on the stop sleeve is indicated in the dosage window;
the dosage setting is locked by sliding the lock collar toward the upper flange until the lock collar actuating mechanism is engaged; and
the set dosage of a substance present in the syringe body is expelled through a dispensing hole at a distal end of the administration tip.

3. The adjustable dose drug delivery system of claim 1, wherein
the mechanism that engages the stop sleeve and outer housing to limit movement of the plunger in accordance with a dosage setting comprises
a plurality of stop sleeve slots comprising indentations in the stop sleeve, the stop sleeve slots deposed at the stop sleeve distal end and extending toward the stop sleeve proximal end for varying distances depending on a specific dosage corresponding to each stop sleeve slot; and
a stop sleeve slot engagement ridge protruding from an inside surface of the outer housing, wherein the stop sleeve slot engagement ridge engages with a stop sleeve slot proximal end to limit movement of the stop sleeve and attached plunger in accordance with the dosage setting.

4. The adjustable dose drug delivery system of claim 1, wherein the mechanism that engages the inner housing and stop sleeve further comprises
a plurality of engagement pits circumferentially and vertically deposed on the inner housing between the inner housing proximal end and the inner housing distal end;
a stop sleeve tongue at a distal end of a stop sleeve slot formed by cutouts at each side of the stop sleeve slot; and
a stop sleeve tongue inner knob protruding inside of a distal end of the stop sleeve tongue,
wherein the stop sleeve tongue inner knob engages an engagement pit to limit movement of the stop sleeve toward the inner housing proximal end.

5. The adjustable dose drug delivery system of claim 1, further comprising components joining the inner housing and the stop sleeve to rotate together.

6. The adjustable dose drug delivery system of claim 5, wherein the components joining the inner housing and the stop sleeve to rotate together comprise
a stop sleeve engagement rail comprising an elevated rail on the inner housing that substantially extends from the inner housing upper flange to the inner housing proximal end; and
an inner housing engagement guide comprising a protruding area at the stop sleeve distal end that accommodates the stop sleeve engagement rail.

7. The adjustable dose drug delivery system of claim 1, wherein the lock collar actuating mechanism comprises
an outer housing flap in the outer housing distal region, the outer housing flap further comprising (a) a lock collar engagement tab the protrudes outward on the outer housing flap, and (b) an outer housing flap inner tab protruding from an inside surface of the outer housing flap;
a lock collar upper shelf comprising a ledge protruding from an inside of the lock collar on a proximal area of the lock collar; and
a dosage lock railing comprising a ledge protruding from a proximal area of the inter-flange area of the inner housing, wherein the dosage lock railing has a gap that accommodates the outer housing flap inner tab, wherein
when the lock collar is slid toward the upper flange, the ledge of the lock collar upper shelf pushes the lock collar engagement tab, directing the outer housing flap inwardly, which causes the outer housing flap inner tab to enter the gap in the dosage lock railing, preventing rotation of the outer housing relative to the inner housing.

8. The adjustable dose drug delivery system of claim 7, further comprising
a lock collar lower shelf comprising a ledge protruding from an inside of the lock collar on a distal area of the lock collar; and
a lock collar guide rail comprising a ledge in the outer housing distal region, wherein
the lock collar lower shelf rests on the lock collar guide rail prior to engagement of the lock collar actuating mechanism.

9. The adjustable dose drug delivery system of claim 1, wherein the plunger proximal end further comprises a cap connector and a snap top, wherein
the snap top is a disk with a central fastener that joins with the cap connector to affix the snap top to the plunger proximal end.

10. The adjustable dose drug delivery system of claim 1, further comprising a seal pin, wherein the seal pin comprises
a seal pin stem comprising a long, thin needle that can be inserted into the administration tip hollow portion through a dispensing hole at a distal end of the administration tip; and
a seal pin top at a distal end of the seal pin stem, wherein the seal pin top prevents complete insertion of the seal pin into the administration tip.

11. The adjustable dose drug delivery system of claim 1, further comprising a protective cap that encloses the administration tip and is affixed to the lower flange.

12. The adjustable dose drug delivery system of claim 1, wherein the administration tip is suitable for rectal administration.

13. The adjustable dose drug delivery system of claim 1, wherein the syringe body comprises diazepam.

14. An adjustable dose drug delivery system comprising:
a plunger, a syringe body, an inner housing, an outer housing, a stop sleeve, a lock collar, a seal pin, and a protective cap, wherein
the plunger comprises a stopper at a plunger distal end, a plunger proximal end, and a plunger stem extending from the plunger distal end to the plunger proximal end, the plunger proximal end further comprises a cap connector and a snap top, wherein
the snap top is a disk with a central fastener that joins with the cap connector to affix the snap top to the plunger proximal end;
the syringe body comprises a cylinder, and further comprises a syringe tip at a syringe body distal end and an open syringe body proximal end comprising a syringe body flange,
wherein the plunger is slidably deposed by inserting the stopper into the open proximal end of the syringe body and pushing the plunger flange toward the syringe body distal end;
the inner housing comprises a cylinder, and further comprises an inner housing proximal end and an inner housing distal end and encloses the syringe body and is affixed thereto, wherein the inner housing further comprises
an upper flange,
a lower flange between the upper flange and the inner housing distal end, with an inter-flange area between the upper flange and lower flange, and
an administration tip distal to the lower flange that is hollow and accommodates, at its proximal end, the syringe tip;
the outer housing comprises a cylinder that encompasses the inner housing from above the lower flange to above the mechanism that engages the stop sleeve, the outer housing further comprising an outer housing proximal end, an outer housing distal end, wherein the outer housing further comprises
an outer housing distal region adjacent to the inter-flange area of the inner housing, wherein the outer housing distal region
has a narrower circumference than the outer housing proximal end, and
comprises a lock collar actuating mechanism that, when engaged, prevents rotation of the outer housing relative to the inner housing;
a dosage window displaying dosage information present on an outer surface of the stop sleeve; and
a stop sleeve engagement mechanism that engages the stop sleeve to prevent the stop sleeve from further movement when a designated dosage is dispensed;
the stop sleeve comprises a cylinder with a stop sleeve proximal end and a stop sleeve distal end, wherein the stop sleeve is deposed between the outer housing and the inner housing above the upper flange,
the stop sleeve further comprising
markings near the stop sleeve distal end indicating dosage settings;
a plurality of stop sleeve slots comprising indentations in the stop sleeve, the stop sleeve slots deposed at the stop sleeve distal end and extending toward the stop sleeve proximal end for varying distances depending on a specific dosage corresponding to each stop sleeve slot; and
a stop sleeve slot engagement ridge protruding from an inside surface of the outer housing, wherein the stop sleeve slot engagement ridge engages with a stop sleeve slot proximal end to limit movement of the stop sleeve and attached plunger in accordance with the dosage setting;
the lock collar encircles a portion of the outer housing distal region and engages with the lock collar actuating mechanism when the lock collar is slid toward the upper flange,
wherein the lock collar actuating mechanism comprises
an outer housing flap in the outer housing distal region, the outer housing flap further comprising (a) a lock collar engagement tab the protrudes outward on the outer housing flap, and (b) an outer housing flap inner tab protruding from an inside surface of the outer housing flap;
a lock collar upper shelf comprising a ledge protruding from an inside of the lock collar on a proximal area of the lock collar; and
a dosage lock railing comprising a ledge protruding from a proximal area of the inter-flange area of the inner housing, wherein the dosage lock railing has a gap that accommodates the outer housing flap inner tab, wherein
when the lock collar is slid toward the upper flange, the ledge of the lock collar upper shelf pushes the lock collar engagement tab, directing the outer housing flap inwardly, which causes the outer housing flap inner tab to enter the gap in the dosage lock railing, preventing rotation of the outer housing relative to the inner housing;
the seal pin comprises
a seal pin stem comprising a long, thin needle that can be inserted into the administration tip hollow portion through a dispensing hole at a distal end of the administration tip; and
a seal pin top at a distal end of the seal pin stem, wherein the seal pin top prevents complete insertion of the seal pin into the administration tip; and
the protective cap encloses the administration tip and is affixed to the lower flange,
the adjustable dose drug delivery system further comprising:
components joining the inner housing and the stop sleeve to rotate together, comprising
a stop sleeve engagement rail comprising an elevated rail on the inner housing that substantially extends from the inner housing upper flange to the inner housing proximal end; and
an inner housing engagement guide comprising a protruding area at the stop sleeve distal end that accommodates the stop sleeve engagement rail;
a lock collar lower shelf comprising a ledge protruding from an inside of the lock collar on a distal area of the lock collar; and
a lock collar guide rail comprising a ledge in the outer housing distal region, wherein
the lock collar lower shelf rests on the lock collar guide rail prior to engagement of the lock collar actuating mechanism,
wherein
the dosage is set by turning the outer housing relative to the stop sleeve, inner housing, syringe body and plunger until a desired dosage marked on the stop sleeve is indicated in the dosage window;
the dosage setting is locked by sliding the lock collar toward the upper flange until the lock collar actuating mechanism is engaged; and
the set dosage of a substance present in the syringe body is expelled through a dispensing hole at a distal end of the administration tip.

15. A method of administering a medicament using the adjustable dose drug delivery system of claim 1, the method comprising
hold the outer housing proximal to the outer housing distal region, grasp the lower flange and turn the lower flange relative to the outer housing until a desired dosage is indicated in the dosage window;
slide the lock collar toward the upper flange to engage the lock collar actuating mechanism;
insert the administration tip into a bodily orifice of a patient in need of the medicament;
dispense the medicament through the administration tip by pushing the plunger toward the syringe body distal end until the plunger cannot be pushed any further.

16. The method of claim 15, further comprising, after dispensing the medicament,
pull the plunger in a proximal direction until the plunger is removed from the syringe body;
replace the plunger into the syringe body and push the plunger toward the syringe body distal end until all of the medicament is dispensed through the administration tip.

17. The method of claim 15, wherein the medicament is benzodiazepine and the bodily orifice is a rectal opening.

18. The method of claim 15, wherein the turning of the lower flange relative to the outer housing and the sliding of the lock collar toward the upper flange is performed by a pharmacist who is not the patient.

19. The method of claim 15, wherein the adjustable dose drug delivery system further comprises the seal pin inserted into the administration tip and the protective cap,
the lower flange is grasped and turned by grasping and turning the administration tip, and
the protective cap and the seal pin is removed before the administration tip is inserted into a bodily orifice of the patient.

20. A method of administering a medicament using the adjustable dose drug delivery system of claim 14, the method comprising
hold the outer housing proximal to the outer housing distal region, grasp the protective cap and turn the protective cap relative to the outer housing until a desired dosage is indicated in the dosage window;
slide the lock collar toward the upper flange to engage the lock collar actuating mechanism;
remove (a) the protective cap from the lower flange, and (b) the seal pin from the administration tip;
insert the administration tip into a bodily orifice of a patient in need of the medicament; and
dispense the medicament through the administration tip by pushing the plunger toward the syringe body distal end until the plunger cannot be pushed any further.

21-23. (canceled)
